(19) United States
(12) Patent Application Publication
(10) Pub. No.: US 2009/0226065 A1
(43) Pub. Date: Sep. 10, 2009

(54) Sampling medical images for virtual histology

(76) Inventor: Dongqing Chen, Setauket, NY (US)

Correspondence Address: F. CHAU & ASSOCIATES, LLC, 130 WOODBURY ROAD, WOODBURY, NY, 11797, US

(21) Appl. No.: 11/664,833

(22) PCT Filed: Oct. 7, 2005
(86) PCT No.: PCT/US05/36093

§ 371 Date: Apr. 6, 2007

Related U.S. Application Data
(60) Provisional application No. 60/617,559, filed on Oct. 9, 2004.

(51) Int. Cl. G06K 9/00; A61B 5/05; G06T 17/00; G06F 17/00

(52) U.S. Cl. 382/131; 600/407; 345/424; 707/104.1

(57) ABSTRACT

A system (300, 400, 800) and method (100, 200) are provided for building a digital sample library of lesions or cancers from medical images, the system (300) including an image scanner (310), image visualization or reviewing equipment (320) in signal communication with the image scanner, a digital sample library database (332), and a network for data communication connected between the library, the reviewing equipment, and the at least one scanner; and the method (100) including acquiring patient medical images (112), detecting target lesions in the acquired patient medical images (114, 116, 118), extracting digital samples (120) of the detected target lesions, collecting pathological and histological results (124, 126) of the detected target lesions, collecting diagnostic results of the detected target lesions (128), performing model selection and feature extraction (122) for each digital sample of a lesion, and storing (130) each extracted digital sample for library evolution.

### CROSS-REFERENCE

This application claims the benefit of U.S. Provisional Application Ser. No. 60/617,559 filed on Oct. 9, 2004 and entitled “System and Method for Building the Library of Digital Tissue and its Application to Lesion Detection and Staging”, which is incorporated herein by reference in its entirety.

### BACKGROUND

Two-dimensional (“2D”) visualization of human organs using medical imaging devices has been widely used for patient diagnosis. Currently available medical imaging devices include computed tomography (“CT”) and magnetic resonance imaging (“MRI”), for example. Three-dimensional (“3D”) images can be formed by stacking and interpolating between two-dimensional pictures produced from the scanning machines. Imaging an organ and visualizing its volume in three-dimensional space is beneficial due to the lack of physical intrusion and the ease of data manipulation. However, the exploration of the three-dimensional volume image must be properly performed in order to fully exploit the advantages of virtually viewing an organ from the inside.

Recent advances in medical imaging technology permit improved tissue contrast within acquired medical images. The improved tissue contrast allows detecting the subtle differences between normal and abnormal, or benign and malignant tissues in the medical images. In addition, the better quality images provide more stable characteristics for digital comparison of virtual samples that are taken out from image series acquired in different periods of time. This makes digital or virtual histology/pathology feasible, and opens opportunities for lesion or tumor staging based on medical images.

The current methods focus on the segmentation of lesion region and extraction of image characteristics from it. They usually use only the images that are acquired at one time or from the same patient. The computer-aided-detection (CAD) technology may use a group of patient images for training to allow the CAD algorithm more robustness to all patient images of the same kind. However, the algorithm will not be able to evolve at the end user site after the CAD application is delivered from the vendor. Usually the CAD algorithm is only for detecting rather than for differentiating pathology/histology types of lesion. For example, the colon CAD algorithm is for detection of polyps in the colon. It cannot tell a user whether the finding is a tabular or hyperplastic polyp, or a carcinoma, for example. That is usually done by a biopsy following a lab test. To avoid the invasive biopsy and costly lab test, a technology to meet the same demand is desired to be finished based only on medical images.

### SUMMARY

These and other drawbacks and disadvantages of the prior art are addressed by a system and method of sampling medical images for virtual histology.

An exemplary method embodiment is provided for building a digital sample library of lesions or cancers from medical images, including acquiring patient medical images, detecting target lesions in the acquired patient medical images, extracting digital samples of the detected target lesions, collecting pathological and histological results of the detected target lesions, collecting diagnostic results of the detected target lesions, performing model selection and feature extraction for each digital sample of a lesion, and storing each extracted digital sample for library evolution. The digital sample in the library includes not only the features that are extracted from the image, but also the pathological and histological data and results.

Another exemplary method embodiment is provided for analyzing a digital sample of a lesion or cancer from at least one medical image by comparing the sample to a pre-built digital sample library, including acquiring patient medical images, detecting target lesions in the acquired patient medical images, extracting a digital sample from a detected target lesion, comparing the digital sample to those in a pre-built digital sample library, determining the pathology or histology type of the lesion, and presenting a virtual pathology or histology report based on the library comparison analysis. The medical image visualization and diagnosis application and the digital sample library may be integrated into a single application and be installed in the same workstation. The image visualization and diagnosis application and the digital sample library may also be two different software applications that are installed in separate hardware that are connected via a network.

An exemplary imaging system embodiment is provided for analyzing a digital sample of a lesion or cancer from medical images by comparing samples to a pre-built digital sample library, the system including at least one image scanner, image visualization or reviewing equipment in signal communication with the at least one image scanner, a digital sample library database, which may be implemented on the image visualization equipment, and a network for data communication connected between the library, the reviewing equipment, and the at least one scanner, wherein the network may be web-based for remote access. When the visualization or reviewing application and the digital sample library are integrated in a single application, the similar applications that run on different host hardware may communicate with each other in order to synchronize the evolution of the library.

The technology of the present disclosure may be used for detection of a lesion, classification of pathological/histological sub-type of the lesion, and lesion surveillance by comparing quantitative measurements of the extracted digital sample to those of typical samples in the library. The quantitative measurements include both those from images and pathology/histology knowledge.

These and other aspects, features and advantages of the present disclosure will become apparent from the following description of exemplary embodiments, which is to be read in connection with the accompanying drawings.

### BRIEF DESCRIPTION OF THE DRAWINGS

The present disclosure teaches sampling medical images for virtual histology in accordance with the following exemplary figures, wherein like elements may be indicated by like reference characters, in which:

FIG. 1 shows a schematic flow diagram for creation and evolution of a digital sample library in accordance with an embodiment of the present disclosure;

FIG. 2 shows a schematic flow diagram for the workflow of a system and method for implementing virtual pathological and histological tests in accordance with an embodiment of the present disclosure;

FIG. 3 shows a schematic block diagram for one kind of network setting for the digital sample library usage or service in accordance with an embodiment of the present disclosure;

FIG. 4 shows a schematic block diagram of a system used to acquire medical images and perform a virtual examination of a human organ in accordance with an embodiment of the present disclosure;

FIG. 5 shows a graphical image diagram for a polyp in the endoluminal view in accordance with an embodiment of the present disclosure;

FIG. 6 shows a graphical image diagram for a polyp digital sample coded in a different shade in the endoluminal view, where the maximum and minimum diameters and volume of the polyp are displayed in accordance with an embodiment of the present disclosure;

FIG. 7 shows a graphical image diagram for a dissected polyp digital sample in a 3D view in accordance with an embodiment of the present disclosure;

FIG. 8 shows a schematic block diagram of a system embodiment based on personal computer bus architecture in accordance with an embodiment of the present disclosure; and

FIG. 9 shows a partial schematic flow diagram for a Ray-Filling algorithm for polyp segmentation in accordance with an embodiment of the present disclosure.

### DETAILED DESCRIPTION OF PREFERRED EMBODIMENTS

The present disclosure teaches sampling medical images for virtual histology and pathology. A system and method are provided for building a library of digital samples of lesions derived from medical images. The system and method have application to virtual pathology and histological analysis. The built library supports a user making a diagnosis or classification of lesion type on a new digital sample.

The virtual histology/pathology technology of the present disclosure may avoid the invasive biopsy and costly lab test, while meeting the same demand to be finished based only on medical images. A software application for this purpose must be self-learning or self-evolving. In that way, the software application may become more accurate or robust by self-enrichment at the end user site without the need from the vendor for source code changing or new version updates. A basic idea in this disclosure is to integrate the digital sample library into the reviewing workstation. When the user uses the system to diagnosis a new patient and extracts a new digital sample, the library is enriched and the detection or classification rules for the lesion/abnormality are optimized based on the newly added information.

The library may be installed within the reviewing workstation. When the user uses the reviewing workstation, the newly extracted digital sample can be compared or matched with that of typical sample representatives in the library. By comparing and matching the typical samples in the library, the library may provide rule-based decisions to support the user diagnosis for the lesion type of the newly extracted samples. When the newly extracted sample's true pathological test result is available, the library is evolved by integrating the new digital sample and its pathological information.

Advances in medical imaging technology have led to images with better tissue contrast than previously feasible. The improved tissue contrast permits detection from the medical images of the subtle differences between normal and abnormal tissues, or benign and malignant tissues. Such images can provide stable characteristics for digital comparison of virtual samples that are extracted from the image series, even when the image series are acquired at different time sections or from different subject.

Exemplary embodiments use digital or virtual histology for lesion or tumor staging based on medical images. A system and method for virtual histology may be applied to an exemplary virtual colonoscopy application, for example.

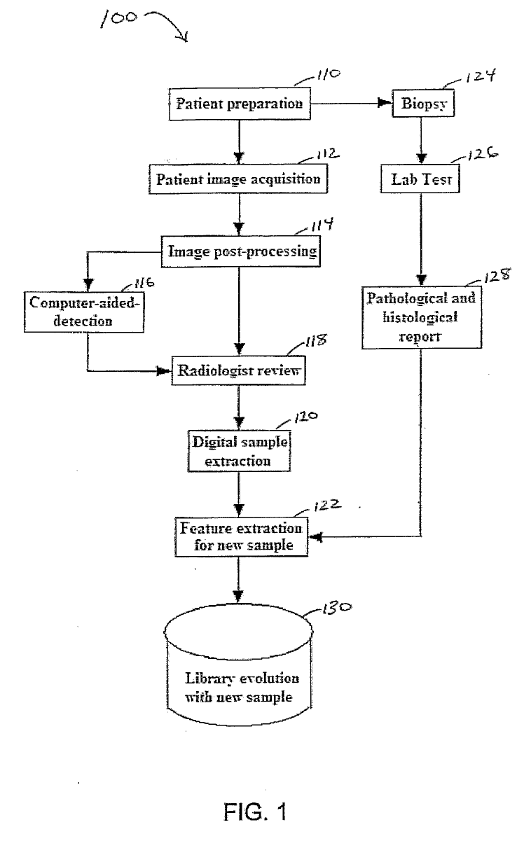

As shown in FIG. 1, a method for creation and evolution of a digital sample library is indicated generally by the reference numeral 100. The method 100 includes a function block 110 that prepares a patient and passes control to a function block 112. The function block 112 performs patient image acquisition and passes control to a function block 114. The function block 114 post-processes the images and passes control to a function block 116 for computer-aided detection, and to a function block 118 for radiologist review. The function block 116 passes control to the function block 118, which, in turn, passes control to a function block 120 to extract digital samples. The function block 120 passes control to a function block 122 to perform feature extraction for each new sample.

The function block 110 also passes control to a function block 124 to perform a biopsy. The function block 124 passes control to a function block 126 to perform a lab test. The function block 126, in turn, passes control to a function block 128 to provide a pathological and histological report. The function block 128 passes this report to the function block 122 for feature extraction. The function block 122 passes the new sample to a database 130 for library evolution with each new sample. Thus, the method 100 demonstrates the workflow of virtual histology. The digital tissue library is a collection of digital samples and their intrinsic characteristics in digital environment.

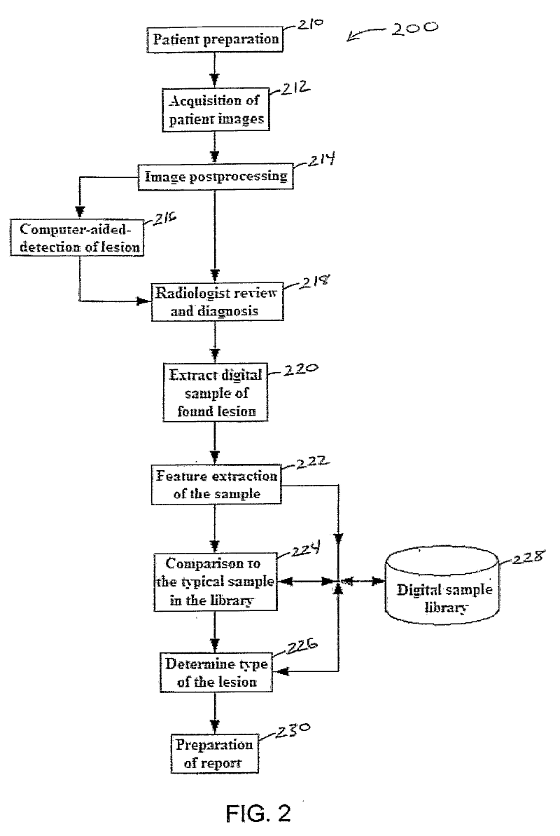

Turning to FIG. 2, a method for implementing virtual pathological and histological tests is indicated generally by the reference numeral 200. The method 200 includes a function block 210 that prepares a patient and passes control to a function block 212. The function block 212 acquires patient images and passes control to a function block 214. The function block 214 post-processes the images and passes control to a function block 216 for computer-aided detection of a lesion, and to a function block 218 for radiologist review and diagnosis. The function block 216 passes control to the function block 218, which, in turn, passes control to a function block 220 to extract digital samples of found lesions. The function block 220 passes control to a function block 222 to perform feature extraction for each new sample. The function block 222 may store sample information in a digital sample library 228.

The function block 222 passes control to a function block 224. The function block 224 receives a typical sample from the digital sample library 228, and compares a found sample to the typical sample from the library. The function block 224, in turn, passes control to a function block 226 to determine the type of lesion. The function block 226 may receive sample feature information from the function block 222 and from the library 228. The function block 228 passes control to a function block 230 for preparation of a report.

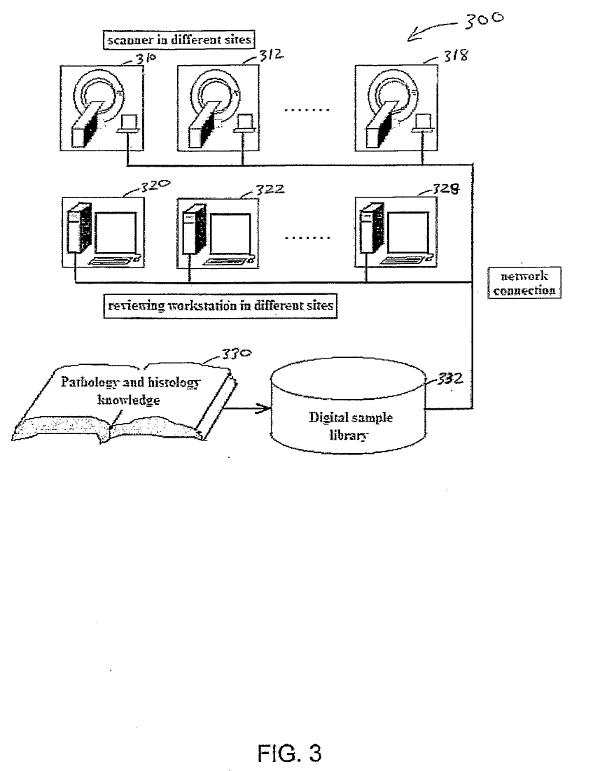

Turning now to FIG. 3, a network with a digital sample library is indicated generally by the reference numeral 300. The network 300 includes scanners 310, 312 and 318, which may be located at different sites. The network 300 further includes reviewing workstations 320, 322 and 328, which may be located at different sites, connected in signal communication with the scanners. Pathology and histology knowledge 330 is supplied to a digital sample library 332, which is connected in signal communication with the scanners 310 through 318 and the reviewing workstations 320 through 328.

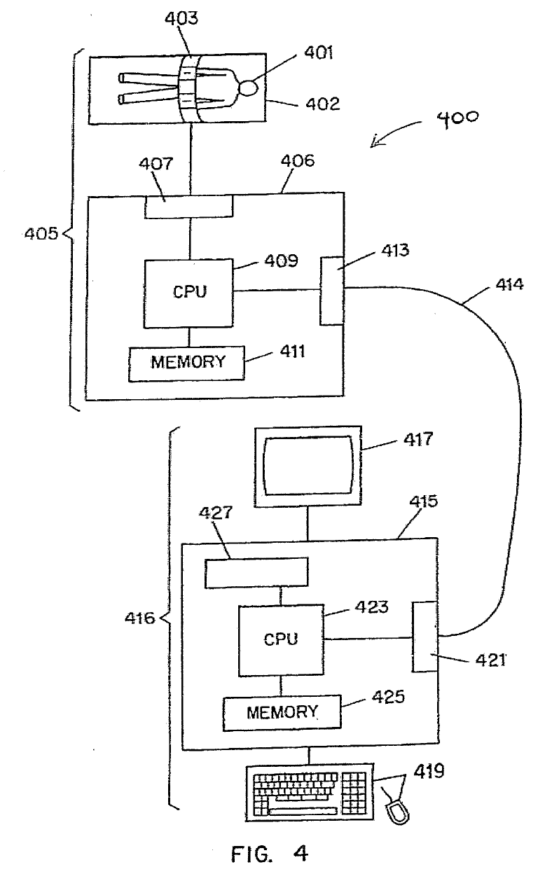

As shown in FIG. 4, a system used to acquire medical images or perform a virtual examination of a human organ in accordance with the disclosure is indicated generally by the reference numeral 400. The system 400 is for performing the virtual examination of an object such as a human organ using the techniques described herein. A patient 401 lays on a platform 402, while a scanning device 405 scans the area that contains the organ or organs to be examined. The scanning device 405 contains a scanning portion 403 that takes images of the patient and an electronics portion 406. The electronics portion 406 includes an interface 407, a central processing unit 409, a memory 411 for temporarily storing the scanning data, and a second interface 413 for sending data to a virtual navigation platform or terminal 416. The interfaces 407 and 413 may be included in a single interface component or may be the same component. The components in the portion 406 are connected together with conventional connectors.

In the system 400, the data provided from the scanning portion 403 of the device 405 is transferred to unit 409 for processing and is stored in memory 411. The central processing unit 409 converts the scanned 2D data to 3D voxel data and stores the results in another portion of the memory 411. Alternatively, the converted data may be directly sent to the interface unit 413 to be transferred to the virtual navigation terminal 416. The conversion of the 2D data could also take place at the virtual navigation terminal 416 after being transmitted from the interface 413. In the preferred embodiment, the converted data is transmitted over a carrier 414 to the virtual navigation terminal 416 in order for an operator to perform the virtual examination. The data may also be transported in other conventional ways, such as storing the data on a storage medium and physically transporting it to terminal 416 or by using satellite transmissions, for example. The scanned data need not be converted to its 3D representation until the visualization-rendering engine requires it to be in 3D form. This saves computational steps and memory storage space.

The virtual navigation terminal 416 includes a screen for viewing the virtual organ or other scanned image, an electronics portion 415 and an interface control 419 such as a keyboard, mouse or space ball. The electronics portion 415 includes an interface port 421, a central processing unit 423, optional components 427 for running the terminal and a memory 425. The components in the terminal 416 are connected together with conventional connectors. The converted voxel data is received in the interface port 421 and stored in the memory 425. The central processing unit 423 then assembles the 3D voxels into a virtual representation and runs a submarine camera model, for example, to perform the virtual examination.

As the submarine camera travels through the virtual organ, a visibility technique may be used to compute only those areas that are visible from the virtual camera, and displays them on the screen 417. A graphics accelerator can also be used in generating the representations. The operator can use the interface device 419 to indicate which portion of the scanned body is desired to be explored. The interface device 419 can further be used to control and move the submarine camera as desired. The terminal portion 415 can be, for example, a dedicated system box. The scanning device 405 and terminal 416, or parts thereof, can be part of the same unit. A single platform would be used to receive the scan image data, connect it to 3D voxels if necessary and perform the guided navigation.

An important feature in system 400 is that the virtual organ can be examined at a later time without the presence of the patient. Additionally, the virtual examination could take place while the patient is being scanned. The scan data can also be sent to multiple terminals, which would allow more than one doctor to view the inside of the organ simultaneously. Thus a doctor in New York could be looking at the same portion of a patient's organ at the same time with a doctor in California while discussing the case. Alternatively, the data can be viewed at different times. Two or more doctors could perform their own examination of the same data in a difficult case. Multiple virtual navigation terminals could be used to view the same scan data. By reproducing the organ as a virtual organ with a discrete set of data, there are a multitude of benefits in areas such as accuracy, cost and possible data manipulations.

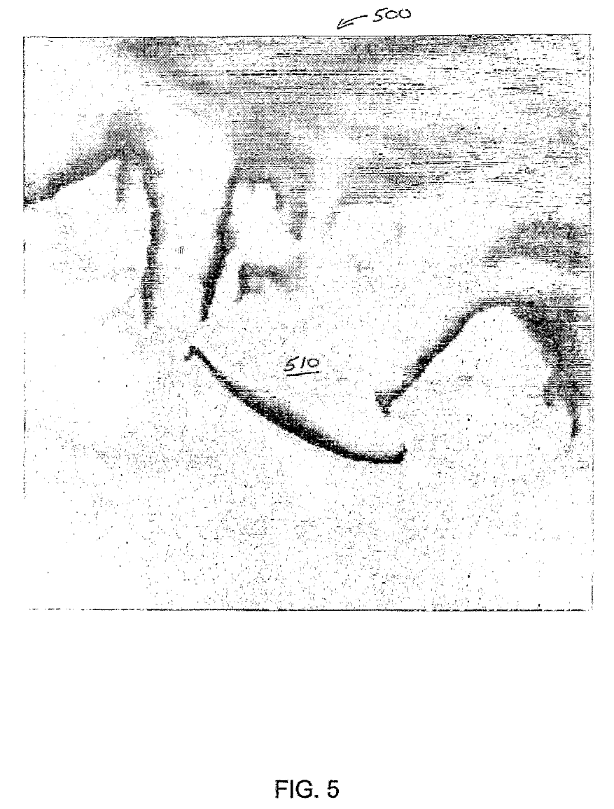

Turning now to FIG. 5, a graphical image is indicated generally by the reference numeral 500. The image 500 includes a polyp 510 in the endoluminal view.

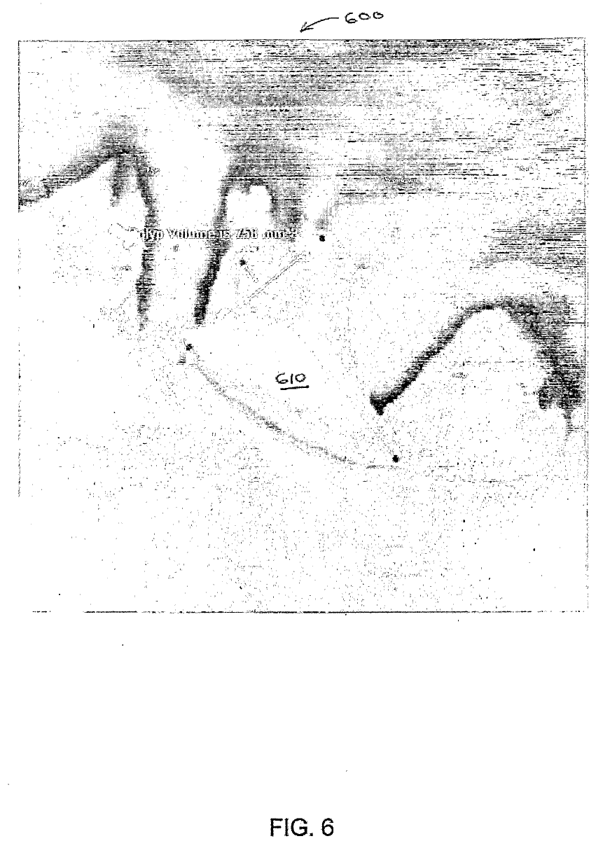

As shown in FIG. 6, a graphical image is indicated generally by the reference numeral 600. The image 600 includes a polyp 610 in the endoluminal view, where the polyp 610 has been digitally sample coded in a different shade. The maximum and minimum diameters and volume of the polyp are displayed in accordance with an embodiment of the present disclosure.

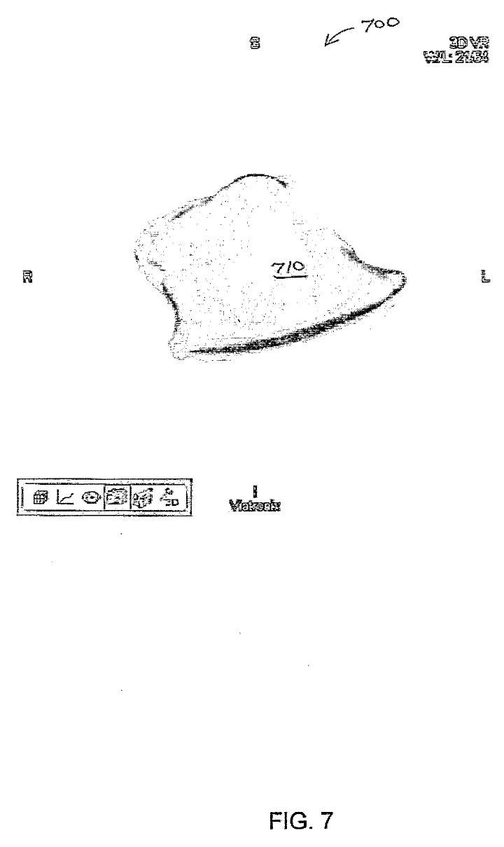

Turning to FIG. 7, a graphical image is indicated generally by the reference numeral 700. The image 700 includes a polyp 710, which is a dissected polyp digital sample in a 3D view.

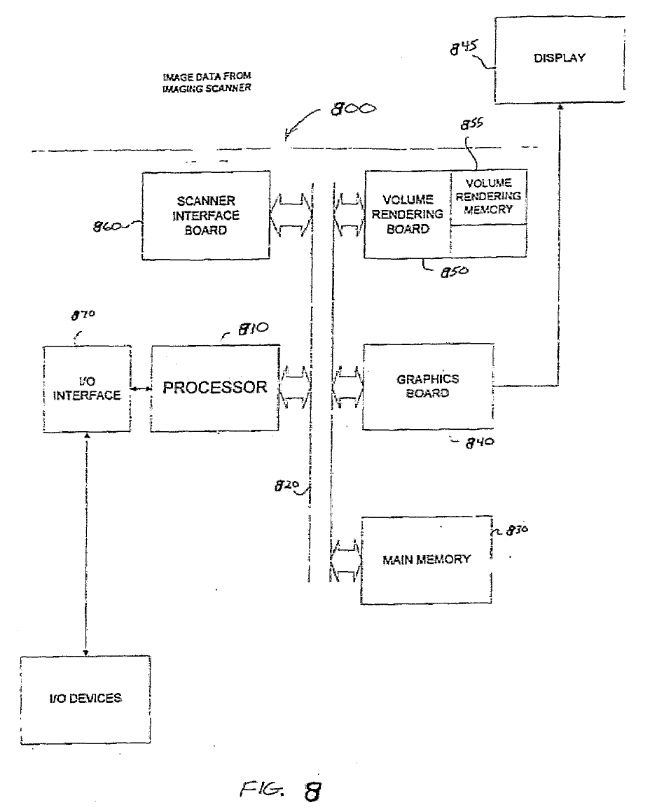

Turning now to FIG. 8, a system embodiment based on personal computer bus architecture is indicated generally by the reference numeral 800. The system 800 includes an alternate hardware embodiment suitable for deployment on a personal computer (PC), as illustrated. The system 800 includes a processor 810 that preferably takes the form of a high speed, multitasking processor. The processor 810 is coupled to a conventional bus structure 820 that provides for high-speed parallel data transfer. Also coupled to the bus structure 820 are a main memory 830, a graphics board 840, and a volume rendering board 850. The graphics board 840 is preferably one that can perform texture mapping. A display device 845, such as a conventional SVGA or RGB monitor, is operably coupled to the graphics board 840 for displaying the image data. A scanner interface board 860 is also provided for receiving data from an imaging scanner, such as an MRI or CT scanner, for example, and transmitting such data to the bus structure 820. The scanner interface board 860 may be an application specific interface product for a selected imaging scanner or can take the form of a general-purpose input/output card. The PC based system 800 will generally include an I/O interface 870 for coupling I/O devices 880, such as a keyboard, digital pointer or mouse, and the like, to the processor 810. Alternatively, the I/O interface can be coupled to the processor 810 via the bus 820.

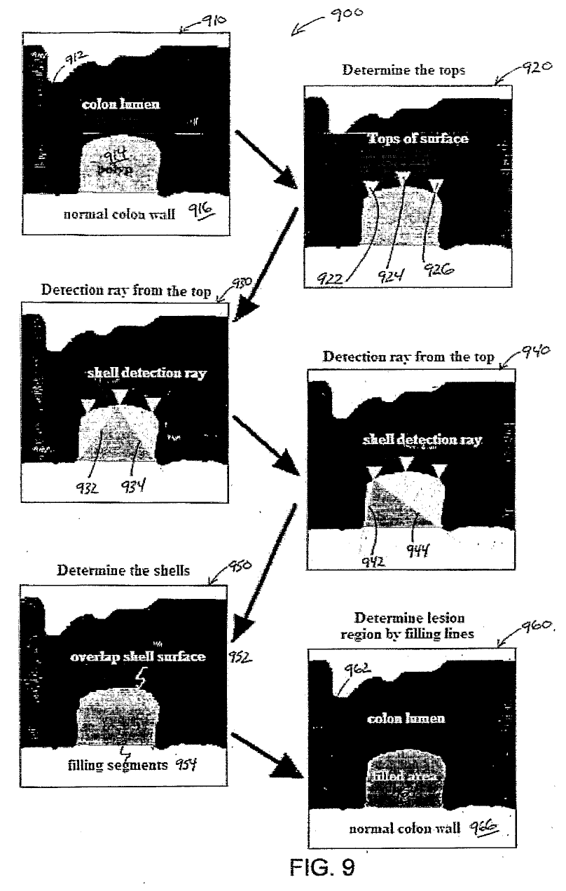

As shown in FIG. 9, a Ray-Filling algorithm for polyp segmentation is indicated generally by the reference numeral 900. The algorithm includes a starting step 910, which shows a colon lumen 912, a polyp 914 encroaching into the lumen, and a normal colon wall 916 disposed beside the lumen and the polyp. A step 920 follows the step 910. The step 920 determines the Tops of the polyp surface, 922, 924 and 926, which are the leftmost, center and rightmost, respectively, and passes control to a step 930. The widest ranging shell detection rays each intersect a point where the lumen 912, polyp 914 and wall 916 meet. The step 930 finds the widest ranging shell detection rays originating from the center Top 924, where a first ray 932 is directed to the left, and a second ray 934 is directed to the right, and passes control to a step 940.

The step 940 finds the widest ranging shell detection rays 942 and 944 originating from the leftmost Top 922 and directed to the left or right, respectively, and passes control to a step 950. The step 950 determines the shells by determining an overlap shell surface 952 and filling segments 954, where the filling segments are segments of all possible line segments with both ends at the overlap shell within the polyp. A step 960 follows the step 950. The step 960 determines a lesion region by filling the area of the filling segments 954 to create a filled area 964 disposed between a colon lumen 962 and a normal colon wall 966.

In operation of the methods 100 and 200 of FIGS. 1 and 2, respectively, a patient may follow a preparation procedure in order to enhance or highlight certain types of tissue or lesions in the images. For example, an intravenous (IV) contrast agent may be used for vessel enhancement in the CT angiograph application. The preparation may be done at a patient's home or at the scanning suite. For example, a patient may orally intake barium for highlighting residues in the colon. In general, the patient preparation may be any kind and may or may not be necessary. The patient preparation for virtual colonoscopy includes the colon lumen distention with room air or CO2 for both CT and MRI scan. For MRI scan, the colon may be filled with warm tap water with or without contrast agent in the water.

A series of medical images is acquired from a subject at a scanning suite after patient preparation. Multiple image series can be acquired based on different patient body positions or on different acquisition sequences in MRI scans. The images can have any modality with high resolution and good tissue contrast. The subject can be a human being or animal, for example. The computer system receives the medical images and post-processes them. The computer system can be directly connected to the image acquisition equipment or connected via a network, such as shown for the system 300 of FIG. 3. The post-processing can have a multiple purpose nature. For example, the purposes may include image enhancement, noise reduction, organ segmentation, initial detection of abnormalities, building of a 3D model for display, and the like.

After post-processing, the images will be loaded and displayed on a medical imaging workstation in various display modes for physician review. The initial results detected by the computer algorithm at the post-processing step will be labeled and may be provided to a physician for diagnosis assistance. After a physician confirms an abnormality, he or she can use a mouse to click on the target. The system will automatically or interactively extract the target sub-volume to encapsulate that abnormality region. The sub-volume is the so-called digital sample for the abnormality. The sub-volume is not a merely group of voxels. It is extracted based on the minimum size for representing a certain lesion or abnormal tissue function. It will provide the basic functional clue for a pathology analysis.

A database of digital samples will be built. The initial digital samples in the database will be used for feature selection. The unique features related to a specific type of abnormality will be extracted for all digital samples of that type. The features are the essential characteristics for the specific type of abnormality. In other words, an indicator of tissue type for that kind of abnormality can be constructed based on those features, and the indicator must have high sensitivity for characterization of the specific abnormality.

The features and the built indicator for a specific tissue type are associated with the digital sample as a whole tissue sample with a certain bio-function, rather than as a group of voxels. This is completely different from that of conventional computer aided) detection (CAD) approaches. In conventional CAD approach, the extracted feature is related to an independent voxel or a group of voxels, where the entire digital sample had never been considered at its feature extraction stage. In other words, the conventional CAD approach works on a collection of fragment information of a tissue type, and tries to put them together to get a conclusion. Instead, the virtual histology technique of the present disclosure works on the complete tissue sample as a whole from the very beginning. The features that are extracted from a digital sample must be global rather than voxel-wise to the tissue type or type of lesion.

For certain lesion types, the initial features and the tissue indicator will be collected and developed in a digital sample library. The digital sample library is a categorized database for features and digital sample indicators. When a new digital sample is obtained, the features that are extracted from the new sample will be compared to those in the library. Using the tissue indicator of the library, one can get a conclusion that the new digital sample is most probably a certain type of known tissue in the library.

Data-mining technology should be employed for improving and enriching the library when more digital samples become available. The digital sample can be stratified in different categories based on type of lesion or different stages of the same type of lesion, such as, for example, benign and malignant polyps.

The consistency of the digital sample is important in terms of its physical characteristics. In other words, the method may assume that the quality of the medical images guarantee that the same tissue type will have similar properties regardless of diverse subjects and acquisition days. This is a basic assumption for the feasibility of virtual histology. In addition to the image quality, the method of extracting digital samples is essential. It must segment out the correct sub-volume in a consistent way with respect to the size, contour, voxel resolution, and the normalized voxel intensity.

An exemplary embodiment method may be adapted to a virtual colonoscopy environment. The image acquisition procedure of virtual colonoscopy can be the routine one as known in the art, for example. The post-processing and display modes for physician review can be any of the available modes. The only thing that triggers the virtual histology is a mouse click in this embodiment. By clicking on the suspicious polyp region, a virtual polypectomy algorithm is applied. The selected sub-volume of the target polyp will be delineated as the digital sample.

The initial suspicious polyp location can be either provided by CAD algorithm or by radiologist manual input. In order to facilitate greater understanding of the exemplary embodiment, the shape feature is used as an example to develop a polyp indicator. Other embodiments are not limited to using only shape features for polyp indicators.

Where a polyp is growing inward to the lumen, its shape is different from those of a Haustral fold and normal colon wall surface. It has a roughly convex or cap-like top with or without a stake. By developing a local intrinsic landmark system on the polyp sub-volume, a shape template can be developed, which should be invariant to translation and rotation. The shape templates that are collected from a training set can be classified to represent polyps of different types, Haustral fold, and normal colonic surface. A library of shape templates will be developed based on available digital samples of polyps. When a new case comes in, the newly collected digital sample will be compared with the templates in the library for tissue confirmation.

As discussed, FIG. 5 shows a polyp in endoluminal view and FIG. 6 shows the extracted digital polyp sample that is coded in a different color in the endoluminal view. The maximum and minimum diameter and its volume are displayed. FIG. 4 shows a digital sample of the dissected polyp that is stored in the library.

Referring back to FIG. 9, the Ray-Filling algorithm for polyp segmentation is designed to automatically delineate the polyp or cancer region from the CT or MR images based on an initial region of the polyp or cancer. In the virtual colonoscopy CT images, the colonic lumen is distended with air or CO2. The air lumen looks dark while the polyp and soft tissue look gray in the CT images. Assuming that a polyp always intrudes into the lumen as a convex cap-shape object, a Ray-Filling algorithm may be used for automatically segmenting the polyp based on a single input point.

The single input point should be at the surface of the polyp. By computing the shape index or curvature features, one can find out all possible convex surface points that are connected to the initial point within the polyp surface shell. This is called the Initial Shell area. From the Initial Shell, three Tops can be determined. Each Top is the point on a region of the shell that is the most convex based on its shape index.

From each Top, rays will be sent out along all directions. The rays start from the Top, which is a soft tissue voxel, and will stop at the first non-soft-tissue point or at the distance bounds. The distance bound is set to the maximum diameter of a possible biggest polyp. Since the polyp surface shell is smooth and continuous, the rays that stop at the distance bounds can be dropped based on the discontinuity of the ray distance. The ending points of the remaining rays form a Secondary Shell, which is usually larger than the Initial Shell. The overlap of all Secondary Shells that are created from different Tops can be determined. This is the Final Shell for the polyp region.

For any two different voxels at the Final Shell, a line segment can be computed. All of the voxels on these line segments can also be determined. Those voxels, as whole, make up the region of the polyp. Since the region is determined by filling the line segment, it is called the Ray-Filling algorithm. The found region is usually a little bit smaller than the true polyp region. A subsequent dilation operation may be combined with morphological knowledge to keep the convexity and allow for a more accurate result.

A method embodiment of the present disclosure is provided for building a digital sample library for certain lesion or cancer in the medical images. This method includes acquiring patient medical images, detecting target lesions in patient images, extracting digital samples of the lesions, collecting pathological and histological results of the lesions, collecting diagnostic results of the lesions, selecting a model and extracting features for the digital sample of a lesion, and storing the digital sample for library evolution when a new digital sample is added.

The method embodiment for building a digital sample library may use acquired patient medical images such as CT, MR, or other modality tomography images. Detection of the lesions may be accomplished with the procedure of radiologists finding the lesion by using a 2D/3D visualization software or system. Detection of the lesions may also be accomplished by a computer-aided-detection software application that detects the findings. Alternatively, radiologists may detect the findings by reviewing concurrently or taking a second look at the list of findings presented by the computer-aided-detection application.

Extracting digital samples of the lesions may further include placing the initial region of the found lesion, automatically labeling the region of the entire lesion covering the initial region, displaying the entire lesion in 2D/3D views for radiologist editing, and extracting the sub-volume that covers the entire lesion with a labeled lesion region. Here, placing the initial region of the found lesion may represent a single mouse-click to point to a voxel in the 2D/3D views. As one alternative, a radiologist manually draws a small 2D/3D region in the 2D images. As another alternative, the computer-aided-detection application automatically marks a voxel or a group of voxels for the initial region of the lesion. Automatically labeling the region of the entire lesion may represent a simple region-growing within a certain range of intensities in the medical images.

Automatically labeling the region of the entire lesion may further include tissue segmentation based on voxel intensity or a group of voxel intensities, application of a Ray-Filling algorithm for delineating a region of lesions within certain tissue areas with the help of the prior knowledge on the lesion morphology, and/or region refinement based on pathological and anatomical knowledge. Radiologist editing of the found lesion region represents that a radiologist may use a 2D/3D painting brush to discard or add regions to the displayed lesion regions. Extraction of the sub-volume that covers the entire lesion may be a parallelepiped, which is centered at the center of the lesion region. The parallelepiped may be aligned and truncated to encompass all necessary morphological, pathological, and histological information that relates to the lesion.

The model selection and feature extraction for the digital sample may further include extracting intensity features for the lesion region, extracting texture features for the lesion region, extracting morphological features for the lesion region, constructing a fused and standardized feature vector, and computation of the representative feature vectors for each pathological and histological type. Here, the intensity feature may include at least average intensity in the lesion region. The morphological feature for the lesion region may include at least the maximum diameter and scattering coefficient. The construction of a fused feature vector can be implemented by normalizing each feature element by its own standard deviation and putting them all together to form a general feature vector. The representative feature vectors can be the mean vector of all vectors coming from the lesion of a particular pathological and histological type.

Pathological and histological results may include tissue type, lesion type, size measurement, benign or malignant, and the like. The diagnostic report may include the lesion location reference to certain human organs or body. The digital sample storing and library evolution may further include constructing a mega data structure for a digital sample, and updating the representative feature vectors for the pathological or histological type if a new digital sample of that type is added in the library. Updating the representative can be implemented by computing the new mean feature vector for a certain pathological or histological lesion type.

Another method embodiment of the present disclosure is provided for analyzing a digital sample of a lesion or cancer from medical images by comparing samples to a pre-built digital sample library. This method includes acquiring patient medical images, detecting the target lesion, extracting a digital sample of the lesion, comparing the digital sample to those in a pre-built digital sample library, determining the pathology or histology type of the lesion, and presenting the virtual pathology or histology report based on the library comparison analysis.

In this embodiment, acquired patient images means acquired patient's CT or MR images with or without contrast agent applied. Detection of a lesion or lesions represents the procedure of radiologists finding the lesion by using a 2D/3D visualization software or system. Detection of a lesion may represent that a computer-aided-detection software application detects the findings. As an alternative, a radiologist detects findings by reviewing concurrently or taking a second look on the list of findings presented by the computer-aided-detection application. Extracting a digital sample of the lesions may further include placing the initial region of the found lesion, automatically labeling the region of the entire lesion covering the initial region, displaying the entire lesion in 2D/3D views for radiologists editing, and extraction of the sub-volume that covers the entire lesion with lesion region labeled.

Placing the initial region of the found lesion may represent a single mouse-click to point to a voxel in 2D/3D views. In an alternative, a radiologist manually draws a small 2D/3D region in the 2D images. In another alternative, the computer-aided-detection application provides a voxel or a group of voxels as an initial region. Automatically labeling the region of the entire lesion may represent a simple region-growing within a certain range of intensities in the medical images. Automatically labeling the region of the entire lesion may further include tissue segmentation based on voxel intensity or a group of voxel intensities, application of a Ray-Filling algorithm for delineating regions of lesions within certain tissue areas with the help of knowledge of lesion morphology, and region refinement based on pathological and anatomical knowledge.

Radiologist editing of the lesion region represents that a radiologist uses a 2D/3D painting brush to discard or add regions to the displayed lesion regions. Extraction of the sub-volume that covers the entire lesion may be a parallelepiped, which is centered at the center of the lesion region. The parallelepiped is aligned and truncated to encompass all necessary morphological, pathological, and histological information that relates to the lesion.

Comparing a digital sample to those in a pre-built digital sample library may further include extracting features of the digital sample and computing the feature vector associated to the sample, transferring the digital sample and feature data to the library server if the library server is running on a different system at different physical location, determining the most similar representative feature vector in the library, and computing the likelihood that the digital sample is likely to be the pathology or histology type that associates to that most similar representative feature vector. Extracting features of the digital sample and computing the feature vector associated to the sample can be employed using any suitable technique, such as those given above. Determining the most similar representative feature vector in the library can employ the Euclidean or Markovian distance between feature vectors as a similarity measure. Computing a likelihood of a sample being a certain pathological or histological type can be implemented by applying the scattering analysis to all available samples of that type in the library.

Determination of the pathological or histological type of the lesion can further apply a Bayesian network method to do the data fusion based on the likelihood of each pathological or histological type. Presenting the virtual pathology or histology report based on the library comparison analysis may further include adding the sample to the library to enrich the library if the true pathology and histology results are available, providing a diagnosis on lesion type, cancer staging, and benign or malignant information with 2D/3D views of the lesion, and providing an electronic diagnosis file including diagnosis information and the digital sample and its sub-volume data for a portable health-care report. Enrichment of the library can employ any combination of the suitable methods that are described above if the true pathological and histological type is later available for the lesion. Providing the electronic diagnosis file can further put all files in a portable device combined with a software application to allow the device to plug-and-play on any regular PC.

An imaging system embodiment of the present disclosure is provided for analyzing digital samples of lesions or cancers from medical images by comparing the samples to a pre-built digital sample library. This system includes image scanners, image visualization equipment, and a database for the digital sample library. It may be implemented on either a visualization apparatus or a separate apparatus. The system also includes a network for data communication between the library, the reviewing equipment, and the scanner. The network may be web-based for remote access.

The image scanner can be CT, MR, Ultrasound, or any 3D tomography scanner for medical use, with a network connection available. The image visualization equipment can be any PC or workstation with a 2D/3D visualization software application installed. The database for the digital sample library can be installed within the visualization equipment or installed on a dedicated server. The server connects to the client visualization equipment via computer network. The network can be the Internet. The network for data communication between the library server and the client visualization equipment can be a local network or via the Internet. The library server can provide service to multiple clients or institutions at different remote physical sites.

Another method embodiment for building a digital sample library for colon polyps, masses, and cancers includes acquiring patient computed tomography colonography (CTC) or magnetic resonance colonography (MRC) images; detecting polyps, masses, and cancers; extracting digital samples of the polyps, masses, and cancers; collecting pathological and histological results of polyps, masses, and cancers; creating a data representation of the digital sample in the library; and enabling the library evolution when the new sample is added.

Another embodiment is provided for analyzing the type of colonic polyps, masses, and the staging of colonic cancers. Here, a method includes acquiring patient CT or MR images; detecting polyps, masses, and cancers; extracting digital samples of the found polyps, masses, or cancers; comparing the digital sample to those in the library in order to determine the pathological or histological type for the polyps, masses, or cancers, and presenting the virtual pathological or histological report.

The foregoing merely illustrates the principles of the disclosure. It will thus be appreciated that those skilled in the art will be able to devise numerous systems, apparatus and methods which, although not explicitly shown or described herein, embody the principles of the disclosure and are thus within the spirit and scope of the disclosure as defined by its Claims.

For example, the methods and systems described herein could be applied to virtually examine an animal, fish or inanimate object. Besides the stated uses in the medical field, applications of the technique could be used to detect the contents of sealed objects that cannot be opened. The technique could also be used inside an architectural structure such as a building or cavern and enable the operator to navigate through the structure.

These and other features and advantages of the present disclosure may be readily ascertained by one of ordinary skill in the pertinent art based on the teachings herein. It is to be understood that the teachings of the present disclosure may be implemented in various forms of hardware, software, firmware, special purpose processors, or combinations thereof.

Most preferably, the teachings of the present disclosure are implemented as a combination of hardware and software. Moreover, the software is preferably implemented as an application program tangibly embodied on a program storage unit. The application program may be uploaded to, and executed by, a machine comprising any suitable architecture. Preferably, the machine is implemented on a computer platform having hardware such as one or more central processing units (“CPU”), a random access memory (“RAM”), and input/output (“I/O”) interfaces. The computer platform may also include an operating system and microinstruction code. The various processes and functions described herein may be either part of the microinstruction code or part of the application program, or any combination thereof, which may be executed by a CPU. In addition, various other peripheral units may be connected to the computer platform such as an additional data storage unit and a printing unit.

It is to be further understood that, because some of the constituent system components and methods depicted in the accompanying drawings are preferably implemented in software, the actual connections between the system components or the process function blocks may differ depending upon the manner in which embodiments of the present disclosure are programmed. Given the teachings herein, one of ordinary skill in the pertinent art will be able to contemplate these and similar implementations or configurations of the present invention.

Although the illustrative embodiments have been described herein with reference to the accompanying drawings, it is to be understood that the present invention is not limited to those precise embodiments, and that various changes and modifications may be effected therein by one of ordinary skill in the pertinent art without departing from the scope or spirit of the present disclosure. All such changes and modifications are intended to be included within the scope of the present invention as set forth in the appended Claims.

What is claimed is:

1. A method (100) for building a digital sample library of lesions or cancers from medical images, the method comprising:
acquiring (112) patient medical images;
detecting (114, 116, 118) target lesions in the acquired patient medical images;
extracting (120) digital samples of the detected target lesions;
collecting (124, 126) pathological and histological results of the detected target lesions;
collecting (128) diagnostic results of the detected target lesions;
performing (1122) model selection and feature extraction for each digital sample of a lesion; and
storing (130) each extracted digital sample in correspondence with its diagnostic, pathological and histological results for library evolution.

2. A method as defined in claim 1 wherein the patient medical images are acquired using computed tomography (CT), magnetic resonance (MR), or other modality tomographic images.

3. A method as defined in claim 1 wherein detection of the lesions represents the procedure of radiologists finding the lesion by using 2D/3D visualization software or systems.

4. A method as defined in claim 1, detecting target lesions comprising at least one of:
using computer-aided-detection (CAD) software to detect the lesion findings; or
asking a radiologist to detect the lesion findings by reviewing concurrently or taking a second look at the list of findings presented by the computer-aided-detection application.

5. A method as defined in claim 4 wherein a radiologist editing the found lesion region uses a 2D/3D painting brush to discard or add regions to the displayed lesion regions.

6. A method as defined in claim 1, extracting digital samples of the lesions further comprising:
placing the initial region of the found lesion;
automatically labeling the region of the entire lesion covering the initial region;
displaying the entire lesion in 2D/3D views for radiologists editing; and
extracting the sub-volume that covers the entire lesion with the lesion region labeled.

7. A method as defined in claim 6, placing the initial region of the found lesion comprising at least one of:
using a single mouse-click to point to a voxel in the 2D/3D views;
a radiologist manually drawing a small 2D/3D region in the 2D images; or
using a computer-aided-detection application to automatically mark a voxel or a group of voxels to be added to the initial region of the lesion.

8. A method as defined in claim 6 wherein automatically labeling the region of the entire lesion represents a simple region-growing within a certain range of intensities in the medical images.

9. A method as defined in claim 6, automatically labeling the region of the entire lesion further comprising:
performing tissue segmentation based on voxel intensity or a group of voxel intensities;
applying a Ray-Filling algorithm for delineating region of lesions within certain tissue areas with the help of the prior knowledge on the lesion morphology; and
region refinement based on pathological and anatomical knowledge.

10. A method as defined in claim 6 wherein extraction of the sub-volume that covers the entire lesion is a parallelepiped, which is centered at the center of the lesion region and aligned and truncated to encompass all necessary morphological, pathological, and histological information that relates to the lesion.

11. A method as defined in claim 1, model selection and feature extraction for the digital sample further comprising:
extracting an intensity feature for the lesion region;
extracting a texture feature for the lesion region;
extracting a morphological feature for the lesion region;
constructing a fused and standardized feature vector; and
computing the representative feature vectors for each pathological and histological type.

12. A method as defined in claim 11 wherein the intensity feature includes an average intensity in the lesion region.

13. A method as defined in claim 11 wherein the morphological feature for the lesion region includes a maximum diameter and a scattering coefficient.

14. A method as defined in claim 11 wherein construction of the fused feature vector is implemented by normalizing each feature element by its own standard deviation and putting them all together to form a general feature vector.

15. A method as defined in claim 11 wherein the representative feature vector is the mean vector of all vectors coming from the lesion of certain pathological and histological type.

16. A method as defined in claim 1 wherein pathological and histological results include tissue type, lesion type, size measurement, and benign or malignant pathology.

17. A method as defined in claim 1 wherein the diagnostic report includes the lesion location reference to certain human organs or body.

18. A method as defined in claim 1 wherein digital sample storing and library evolution further comprises:
constructing a mega data structure for a digital sample; and
updating the representative feature vectors for the pathological or histological type if a new digital sample of that type is added in the library.

19. A method as defined in claim 18 wherein updating the representative feature vectors is implemented by computing the new mean feature vector for a certain pathological or histological lesion type.

20. A method (200) for analyzing a digital sample of a lesion or cancer from at least one medical image by comparing the sample to a pre-built digital sample library, the method comprising:
acquiring (212) patient medical images;
detecting (214, 216, 218) target lesions in the acquired patient medical images;
extracting (220) a digital sample from a detected target lesion;
comparing (224) the digital sample to those in a pre-built digital sample library;
determining (226) the pathology or histology type of the lesion; and
presenting (230) a virtual pathology or histology report based on the library comparison analysis;
wherein the digital samples in the library each comprise at least one voxel in correspondence with pathology or histology type information.

21. A method as defined in claim 20 wherein acquired patient images are images acquired from a patient's computed tomography (CT) or magnetic resonance (MR) images with or without an applied contrast agent.

22. A method as defined in claim 20 wherein detection of lesion includes the procedure of radiologists finding the lesion by using a 2D/3D visualization software package or system.

23. A method as defined in claim 20 wherein detection of a lesion includes at least one of:
a computer-aided-detection software application detecting the lesion findings; or
a radiologist detecting the lesion findings by reviewing concurrently or taking a second look on the list of findings presented by the computer-aided-detection application.

24. A method as defined in claim 20, extracting a digital sample of a lesion further comprising:
placing the initial region of the found lesion;
automatically labeling the region of the entire lesion covering the initial region;
displaying the entire lesion in 2D/3D views for radiologist editing; and
extracting a sub-volume that covers the entire lesion with the lesion region labeled.

25. A method as defined in claim 24, placing the initial region of the found lesion including at least one of:
using a single-mouse-click to point to a voxel in 2D/3D views;
a radiologist manually drawing a small 2D/3D region in the 2D images; or
using a computer-aided-detection application to provide a voxel or a group of voxels as an initial region.

26. A method as defined in claim 24 wherein automatically labeling the region of the entire lesion includes a simple region-growing process within a certain range of intensities in the medical images.

27. A method as defined in claim 24, automatically labeling the region of the entire lesion further comprising:
tissue segmentation based on voxel intensity or a group of voxel intensities;
application of a Ray-Filling algorithm for delineating a region of lesions within certain tissue areas with the help of knowledge of lesion morphology; and
region refinement based on pathological and anatomical knowledge.

28. A method as defined in claim 24, radiologist editing of the lesion region comprising a radiologist's use of a 2D/3D painting brush to discard or add regions to the displayed lesion regions.

29. A method as defined in claim 24 wherein the extraction of the sub-volume that covers the entire lesion is a parallelepiped, which is centered at the center of the lesion region, aligned and truncated to encompass all necessary morphological, pathological, and histological information that relates to the lesion.

30. A method as defined in claim 20, comparing the digital sample to those in a pre-built digital sample library further comprising:
extracting features of the digital sample and computing the feature vector associated with the sample;
transferring the digital sample and feature data to the library server even if the library server is running on a different system at different physical location;
determining the most similar representative feature vector in the library; and
computing the likelihood that the digital sample is likely to be the pathology or histology type that associates with that most similar representative feature vector.

31. A method as defined in claim 30, extracting features of the digital sample and computing the feature vector associated to the sample comprising:
extracting an intensity feature for the lesion region;
extracting a texture feature for the lesion region;
extracting a morphological feature for the lesion region;
constructing a fused and standardized feature vector; and
computing the representative feature vectors for each pathological and histological type.

32. A method as defined in claim 30 wherein determining the most similar representative feature vector in the library employs the Euclidean or Markovian distance between the feature vectors as a similarity measure.

33. A method as defined in claim 30 wherein computing the likelihood of a sample having a certain pathological or histological type is implemented by applying the scattering analysis to all available samples of that type in the library.

34. A method as defined in claim 20 wherein determination of the pathological or histological type of the lesion further applies a Bayesian network method to do the data fusion based on the likelihood for each pathological or histological type.

35. A method as defined in claim 20, presenting the virtual pathology or histology report based on the library comparison analysis further comprising:
adding the sample to the library to enrich the library if the true pathology and histology results are available;
providing a diagnosis on lesion type, cancer staging, and benign or malignant information with 2D/3D views of the lesion;
providing an electronic diagnosis file including diagnosis information and the digital sample and its sub-volume data for a portable health-care report.

36. A method as defined in claim 35, enrichment of the library if the true pathological and histological becomes available for the lesion comprising:
extracting an intensity feature for the lesion region;
extracting a texture feature for the lesion region;
extracting a morphological feature for the lesion region;
constructing a fused and standardized feature vector; and
computing the representative feature vectors for each pathological and histological type.

37. A method as defined in claim 35 wherein providing the electronic diagnosis file further puts all such files in a portable device combined with a software application to allow the device to plug-and-play on any standard PC.

38. An imaging system (300) for analyzing a digital sample of a lesion or cancer from medical images by comparing samples to a pre-built digital sample library, the system comprising:
at least one image scanner (310);
image visualization or reviewing equipment (320) in signal communication with the at least one image scanner;
a digital sample library database (332), which may be implemented on the image visualization equipment; and
a network for data communication connected between the library, the reviewing equipment, and the at least one scanner, wherein the network may be web-based for remote access;
wherein the database for the digital sample library is installed within the visualization equipment.

39. A system as defined in claim 38 wherein the image scanner is one of a computed tomography (CT), magnetic resonance (MR), ultrasound, or any 3D tomography scanner for medical use with an available network connection.

40. A system as defined in claim 38 wherein the image visualization equipment is any PC or workstation with a 2D/3D visualization software application installed.

41. (canceled)

42. A system as defined in claim 38, further comprising:
second image visualization equipment in signal communication with a second image scanner; and
a second digital sample library database implemented on the second image visualization equipment,
wherein the database for the second digital sample library is installed within the second image visualization equipment, which connects to the first visualization equipment with the network.

43. A system as defined in claim 38 wherein the network for data communication between the library server and the client visualization equipment is selected from a local network or the Internet.

44. A system as defined in claim 38 wherein the library server is disposed for providing service to multiple clients or institutions at different remote physical sites.

45. A method as defined in claim 1, further comprising:acquiring patient CTC or MRC images;
detecting colon polyps, masses, or cancers in the acquired images; and
extracting a digital sample of each detected colon polyp, mass, or cancer.

46. A method as defined in claim 45, further comprising:
collecting pathological and histological results of the detected polyps, masses, or cancers;
creating a data representation of the extracted digital sample in a library; and
enabling evolution of the library for each extracted digital sample.

47. A method for building a digital sample library for colon polyps, masses, and cancers, the method comprising:
acquiring patient CTC or MRC images;
detecting polyps, masses, or cancers in the acquired images;
extracting a digital sample of each detected polyp, mass, or cancer;
collecting pathological and histological results corresponding to the detected polyps, masses, or cancers;
creating a data representation of the extracted digital sample and corresponding results in a library;
enabling evolution of the library for each extracted digital sample;
comparing the extracted digital sample to those in the library in order to determine the pathological or histological type for the polyps, masses, or cancers; and
presenting a virtual pathological or histological report responsive to the comparison and corresponding results.

48. A program storage device readable by machine, tangibly embodying a program of instructions executable by the machine to perform program steps for building a digital sample library of lesions or cancers from medical images, the program steps comprising:
acquiring patient medical images;
detecting target lesions in the acquired patient medical images;
extracting digital samples of the detected target lesions;
collecting pathological and histological results of the detected target lesions;
collecting diagnostic results of the detected target lesions;
performing model selection and feature extraction for each digital sample of a lesion; and
storing each extracted digital sample in correspondence with its diagnostic, pathological and histological results for library evolution.
